(19) United States
(12) Patent Application Publication
(10) Pub. No.: US 2011/0152375 A1
(43) Pub. Date: Jun. 23, 2011

(54) METHOD OF REDUCING THE METHANE GAS LEVEL AND OF INCREASING THE TOTAL GAS YIELD IN ANIMAL FEED

(75) Inventors: Arnulf Tröscher, Weinheim (DE); Michael Koch, Speyer (DE); Natalia Trukhan, Ludwigshafen (DE); Ulrich Müller, Neustadt (DE)

(73) Assignee: BASF SE, Ludwigshafen (DE)

(21) Appl. No.: 12/688,963

(22) Filed: Jan. 18, 2010

(30) Foreign Application Priority Data
Dec. 18, 2009 (DE) 202009017307.0

(51) Int. Cl. A61K 31/19; A23K 1/18; A23K 1/16; A61P 1/00

(52) U.S. Cl. 514/578; 426/648

(57) ABSTRACT

The present invention relates to the use of at least one porous metal-organic framework material (MOF) comprising at least one first and, if appropriate, one second organic compound, where at least the first organic compound binds coordinatively to at least one metal ion in an at least partly bidentate manner, where the at least one metal ion is Mg(II) and where the first organic compound is derived from formic acid and the second organic compound from acetic acid, for reducing the methane level in the total gas produced, and to the use for increasing the total gas formation during feed digestion in ruminants as well as a method for reducing the methane level in the total gas produced and a method for increasing the total gas formation during feed digestion in ruminants.

### CROSS-REFERENCE TO RELATED APPLICATIONS

This claims benefit of German application 202009 017 307.0, filed Dec. 18, 2009 which is incorporated by reference in its entirety for all useful purposes.

### BACKGROUND OF THE INVENTION

Livestock keeping is globally the largest cause of the greenhouses gases caused by man, among which methane accounts for the largest proportion. Each year, all ruminants and livestock produce, in their stomachs, approximately 80 million tones of methane gas, which means not only a contribution to global warming, but also an energy loss of 2-12% for the animal in terms of the amount of energy consumed. The potential of methane with regard to global warming is approximately 21 times higher than that of CO₂. However, methane has a relatively short life span in the atmosphere. The European Union has pledged to reduce the greenhouse gas emissions and has pledged a 20% reduction by 2020. Methane in the stomach is predominantly a by-product of the anaerobic digestion in the stomach. The generation of methane as such cannot be eliminated from the ruminant's metabolic system, but may be manipulated to a certain extent.

There are currently known methods of reducing the methane emission of cattle, using soya oil as a feed additive (University College Dublin), the mechanism on which this method is based still being the subject of studies. A disadvantage of this method is the additional, not inconsiderable costs for the soya oils employed in the animal feed, and, in some cases, adverse effects on crude fiber digestibility.

A further method is to influence the grass species; a higher proportion of leaf in the grass in comparison with a grass with a higher proportion of stalks can reduce the methane emission in cattle (Boland et. al. 2009, Proceedings of American Society of Animal Science, Annual Meeting Montreal). The disadvantage of the method is that it can be used primarily in summer while the animals are at pasture, but not during winter time, or when housed indoors all year round.

### BRIEF SUMMARY OF THE INVENTION

It was therefore an object of the present invention to provide a feed additive or a method which reduces the energy loss caused by methane produced in livestock. A further object was the improvement of the energy yield.

This object was achieved by the use of at least one porous metal-organic framework material (MOF) comprising at least one first and, if appropriate, one second organic compound, where at least the first organic compound binds coordinatively to at least one metal ion in an at least partly bidentate manner, where the at least one metal ion is Mg(II) and where the first organic compound is derived from formic acid and the second organic compound from acetic acid, for reducing the methane level in total gas produced during the feed digestion of a defined feed quantity of a standard feed in ruminants.

Surprisingly, the use of the MOF leads to a methane level in the total gas produced which is 10-15% lower in comparison with the same feed quantity without additive.

### A BRIEF DESCRIPTION OF THE FIGURE

FIG. 1 shows the x-ray diffractogram of the metal-organic framework material according to the invention made of formate and acetate. In the diffractogram, I describes the intensity (Lᵢₙ (counts)) and 2 Θ the 2-theta scale.

FIG. 2 shows the methane level based on the total amount of fermentation gas for MOF magnesium formate with a Langmuir surface of 350 m²/g (P3) and 500 m²/g (P2). The control C was carried out without the addition of magnesium formate, the method being otherwise identical.

FIG. 3 shows the total amount in ml of gas produced during digestion for the two MOF magnesium formate with a Langmuir surface of 350 (P3) and, 500 m²/g (P2) respectively. The control C was carried out without the addition of magnesium formate, the method being otherwise identical.

FIG. 4 shows the amount of methane in ml produced for the two MOF magnesium formate with a Langmuir surface of 350 (P3) and, 500 m²/g (P2) respectively. The control C was carried out without the addition of magnesium formate, the method being otherwise identical.

### DETAILED DESCRIPTION OF THE INVENTION

For the purposes of the present invention, the expression “to derive” is understood as meaning that formic acid and, if appropriate, acetic acid are present in accordance with the present invention in the porous metal-organic framework material in the form of formate or acetate, a protonated form also being possible to some extent.

When a magnesium formate metal-organic framework material is prepared in the presence of acetic acid, it has emerged that a metal-organic framework material can be obtained whose framework structure is comparable to that of the straight magnesium formate framework material.

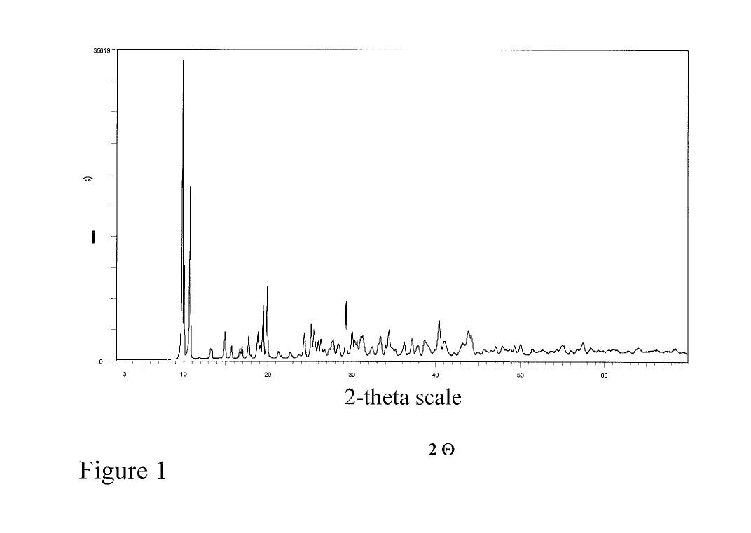

FIG. 1 shows the x-ray diffractogram of the metal-organic framework material made of formate and acetate. In the diffractogram, I describes the intensity (Lᵢₙ (counts)) and 2 Θ the 2-theta scale.

The framework material according to the invention is preferably characterized in that its X-ray diffractogram (XRD) has two reflections in the range from 8°<2Θ<12°, which show the strongest reflections in the range from 2°<2Θ<70°.

The diffractogram can be determined as follows: the sample is installed as powder in the sample container of a commercially available instrument (Siemens D-5000 diffractometer or Bruker D8-Advance). CuαKα radiation with variable primary and secondary orifice plates and a secondary monochromator is used as radiation source. The signal is detected by means of a scintillation counter (Siemens) or Solex semiconductor detector (Bruker). The measurement range for 2Θ is typically from 2° to 70°. The angle step is 0.02°, and the measurement time per angle step is typically 2-4 s. In the evaluation, reflections are indicated by a signal strength which is at least 3 times higher than the background noise. The area analysis can be carried out manually by drawing a baseline on the individual reflections. As an alternative, programs such as “Topas-Profile” from Bruker can be used, in which case the fitting to the background is then preferably carried out automatically by means of a 1st order polynomial in the software.

It is furthermore preferred that the metal-organic framework material according to the invention does not comprise any further metal ions besides Mg(II).

Moreover, it is also preferred that the metal-organic framework material according to the invention does not comprise any further at least bidentate organic compounds which bind coordinatively to the at least one metal ion.

The molar ratio of first to second organic compound in the metal-organic framework material according to the invention is preferably in the range of from 10:1 to 1:10. More preferably, the ratio is in the range of from 5:1 to 1:5, even more preferably in the range of from 2:1 to 1:2, even more preferably in the range of from 1.5:1 to 1:1.5, even more preferably in the range of from 1.2:1 to 1:1.2, even more preferably in the range of from 1.1:1 to 1:1.1 and in particular at 1:1. Accordingly, the amounts of formic acid and acetic acid required in the preparation may be employed.

The metal-organic framework material can be obtained by a process comprising the steps:

reacting a reaction solution comprising magnesium nitrate hexahydrate, formic acid and acetic acid and a solvent at a temperature in the range of from 110° C. to 150° C. for at least 10 hours, and
separating the solid which has precipitated.

The process for the preparation of the framework material according to the invention comprises, as step (a), reacting a reaction solution comprising magnesium nitrate hexahydrate and formic acid, acetic acid and a solvent at a temperature in the range of from 110° C. to 150° C. for at least 10 hours.

The reaction is preferably carried out with stirring, at least for some time, in particular at the beginning of the reaction process.

One starting compound employed is magnesium nitrate hexahydrate. Its initial concentration in the reaction solution is preferably in the range of from 0.005 mol/l to 0.5 mol/l. The initial concentration is furthermore preferably in the range of from 0.1 mol/l to 0.4 mol/l. In particular, the initial concentration is in the range of from 0.15 mol/l to 0.3 mol/l.

In this context, the amount of magnesium nitrate hexahydrate is fed in such an amount to the reaction solution that the magnesium concentration in the reaction solution decreases due to the precipitated solid in step (b).

Moreover, it is preferred that the ratio of the initial amount of formic acid and acetic acid employed to the initial amount of magnesium nitrate hexahydrate is in the range of from 2.5:1 to 3.0:1. Furthermore preferably, the ratio is in the range of from 2.6:1 to 2.9:1, furthermore preferably in the range of from 2.7:1 to 2.8:1. In this context, the total of the initial amounts of formic acid and acetic acid must be taken into consideration accordingly.

Besides magnesium nitrate hexahydrate and formic acid and acetic acid, the reaction solution for step (a) of the process according to the invention for the preparation of the metal-organic framework material according to the invention furthermore comprises a solvent. The solvent must be suitable for dissolving the starting materials employed, at least to some extent. Moreover, the solvent must be chosen such that the temperature range required can be adhered to.

Thus, the reaction in the process according to the invention for the preparation of the material according to the invention is carried out in the presence of a solvent. In this context, solvothermal conditions may be used. The expression “thermal” is understood as meaning, for the purposes of the present invention, a preparation process in which the reaction is carried out in a pressure vessel which is closed during the reaction and to which elevated temperature is applied so that a pressure builds up within the reaction medium as a result of the vapor pressure of the solvent present. As the case may be, the desired reaction temperature may be reached thereby.

Preferably, the reaction is not carried out in water-comprising medium and also not under solvothermal conditions.

Accordingly, the reaction in the process according to the invention is preferably carried out in the presence of a nonaqueous solvent.

The reaction is preferably carried out at a pressure of no more than 2 bar (absolute). However, the pressure is preferably no more than 1230 mbar (absolute). The reaction particularly preferably takes place at atmospheric pressure. However, slight superatmospheric or subatmospheric pressures may occur due to the apparatus. For the purposes of the present invention, the expression “atmospheric pressure” therefore means the pressure range given by the actual atmospheric pressure ±150 mbar.

The reaction takes place in a temperature range of from 110° C. to 150° C. The temperature is preferably in the range of from 115° C. to 130° C. The temperature is furthermore preferably in a range of from 120° C. to 125° C.

The reaction solution may furthermore comprise a base. By using an organic solvent, it is frequently not necessary to employ such a base. Nevertheless, the solvent for the process according to the invention can be selected such that it itself is basic, but this is not absolutely necessary for carrying out the process according to the invention.

It is likewise possible to use a base. However, it is preferred that no additional base is used.

It is furthermore advantageous for the reaction to be able to take place with stirring, which is also advantageous in a scale-up.

The (nonaqueous) organic solvent is preferably a C₁₋₆-alkanol, dimethyl sulfoxide (DMSO), N,N-dimethylformamide (DMF), N,N-diethylformamide (DEF), N,N-di-methylacetamide (DMAc), acetonitrile, toluene, dioxane, benzene, chlorobenzene, methyl ethyl ketone (MEK), pyridine, tetrahydrofuran (THF), ethyl acetate, optionally halogenated C₁₋₂₀₀-alkane, sulfolane, glycol, N-methylpyrrolidone (NMP), gamma-butyrolactone, alicyclic alcohols such as cyclohexanol, ketones such as acetone or acetylacetone, cycloketones such as cyclohexanone, sulfolene or mixtures of these.

A C₁₋₆-alkanol refers to an alcohol having 1 to 6 C atoms. Examples are methanol, ethanol, n-propanol, i-propanol, n-butanol, i-butanol, t-butanol, pentanol, hexanol and mixtures of these.

An optionally halogenated C₁₋₂₀₀-alkane refers to an alkane having 1 to 200 C atoms, it being possible for one or more up to all hydrogen atoms to be replaced by halogen, preferably chlorine or fluorine, in particular chlorine. Examples are chloroform, dichloromethane, tetrachloromethane, dichloroethane, hexane, heptane, octane and mixtures of these.

Preferred solvents are DMF, DEF, DMAc and NMP. DMF is especially preferred.

The expression “nonaqueous” preferably refers to a solvent which does not exceed a maximum water content of 10% by weight, more preferably 5% by weight, furthermore more preferably 1% by weight, furthermore preferably 0.1% by weight, especially preferably 0.01% by weight, based on the total weight of the solvent.

The maximum water content during the reaction is preferably 10% by weight, more preferably 5% by weight and furthermore more preferably 1% by weight.

The expression “solvent” refers to pure solvents and to mixtures of different solvents.

Step (a) of the process according to the invention for the preparation of the framework material according to the invention is carried out for at least 10 hours. Preferably, the reaction takes place for at least one day, more preferably for at least two days.

Furthermore, the process according to the invention features step (b), removal of the precipitated solid.

Due to step (a) of the preparation process according to the invention, the framework material precipitates from the reaction solution as a solid. Removal is carried out by methods known in the prior art, such as filtration or the like.

The porous metal-organic framework material based purely on magnesium formate can be obtained in accordance with the process carried out above or in accordance with the synthesis as described in J. A. Rood et al., Inorg. Chem. 45 (2006), 5521-5528.

The methane content in the fermentation gas is determined as described in the Hohenheim feeds evaluation test (see Examples).

It is advantageous to employ 0.001-10 000 ppm of MOF, preferably 0.01-1000 ppm and in particular 0.1-100 ppm of MOF per kg of feed.

According to one embodiment, the porous metal-organic framework material (MOF) is magnesium formate. It is also feasible that a magnesium formate/acetate MOF is used.

Advantageously, the Langmuir surface of the metal-organic framework material is at least 350 m²/g, preferably 350-500 m²/g, in particular approximately 500 m²/g. If the specific Langmuir surface amounts to only a few square meters and is therefore too low, no effect of the metal-organic framework material can be detected.

The object is furthermore achieved by the use of at least one porous metal-organic framework material comprising at least one first and, if appropriate, one second organic compound, where at least the first organic compound binds coordinatively to at least one metal ion in an at least partly bidentate manner, where the at least one metal ion is Mg(II) and where the first organic compound is derived from formic acid and the second organic compound from acetic acid, for increasing the total gas formation during the feed digestion of a defined feed quantity of a standard feed in ruminants.

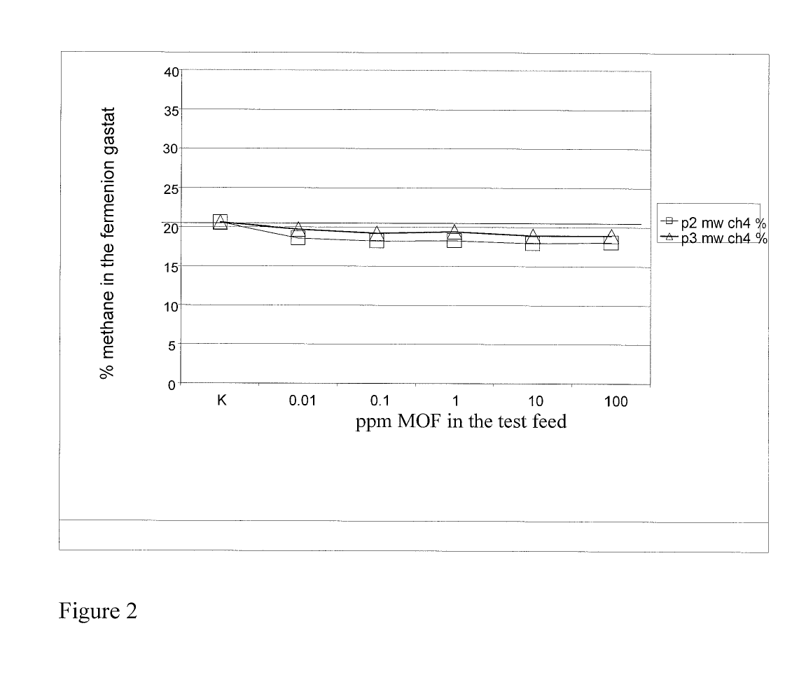

Surprisingly, the use according to the invention of the porous metal-organic framework material leads, with the same amount of feed, to a pronounced increase in the fermentation gas formed of approximately 20%, from approximately 23 ml to approximately 27 ml in a traditional digestion method (see FIG. 2). This increase in the total gas formation means better digestibility of the organic substances fed, whereby an identical performance is achieved with less feed, or less methane is formed for the same performance. Thus, less feed and better digestibility by using the method according to the invention reduces the amount of emitted methane while maintaining the same growth capacity of the cattle.

The relationship between the total amount of gas produced and the energy yield, i.e. the content of metabolizable energy in MJ ME per kg of feed, from a ruminant feed is described in Menke et al. J. agric. Sci. Camb. 1979, 93, 217-222.

The fermentation gas is determined as specified in the Hohenheim feeds evaluation.

The amount of MOF employed in the method according to the invention advantageously amounts to 0.001-10 000 ppm, preferably to 0.01-1000 ppm and in particular to 0.1-100 ppm per kg of feed.

According to one embodiment, the porous metal-organic framework material (MOF) is magnesium formate. It is therefore also feasible to use a magnesium formate/acetate MOF.

In accordance with a particular embodiment, the specific Langmuir surface of the metal-organic framework material is to at least 350 m²/g, preferably 350-500 m²/g, and in particular approximately 500 m²/g. If the specific Langmuir surface amounts to only a few square meters and is therefore too low, no effect of the metal-organic framework material can be detected.

The object is furthermore achieved by a method of reducing the methane level in the total gas produced in ruminants during the feed digestion of a defined feed quantity of a standard feed, comprising the feeding, to a ruminant, of at least one porous metal-organic framework material comprising at least one first and, if appropriate, one second organic compound, where at least the first organic compound binds coordinatively to at least one metal ion in an at least partly bidentate manner, where the at least one metal ion is Mg(II) and where the first organic compound is derived from formic acid and the second organic compound from acetic acid.

Surprisingly, the methane level in the total gas is reduced by approximately 10-15% by the method according to the invention in comparison with the same feed quantity which undergoes traditional digestion.

The methane content in the fermentation gas is determined as specified in the Hohenheim feeds evaluation (VDLUFA, Methodenbuch [Methods Book] volume III, chapter 25.1).

The amount of MOF employed in the method according to the invention advantageously amounts to 0.001-10 000 ppm, preferably to 0.01-1000 ppm and in particular to 0.1-100 ppm per kg of feed.

According to one embodiment, the porous metal-organic framework material (MOF) is magnesium formate. It is also feasible to use a magnesium formate/acetate MOF.

According to a particular embodiment, the specific Langmuir surface of the metal-organic framework material is at least 350 m²/g, preferably 350-500 m²/g, and in particular approximately 500 m²/g. If the specific Langmuir surface amounts to only a few square meters and is therefore too low, no effect of the metal-organic framework material can be detected.

The object is furthermore achieved by a method of increasing the total gas formation in ruminants during the feed digestion, comprising the feeding, to a ruminant, of at least one porous metal-organic framework material comprising at least one first and, if appropriate, one second organic compound, where at least the first organic compound binds coordinatively to at least one metal ion in an at least partly bidentate manner, where the at least one metal ion is Mg(II) and where the first organic compound is derived from formic acid and the second organic compound from acetic acid.

Surprisingly, the method according to the invention leads, with the same amount of feed, to a pronounced increase in the fermentation gas formed of approximately 20%, from approximately 23 ml to approximately 27 ml in a traditional digestion method (see FIG. 2). This increase in the total gas formation means better digestibility of the organic substances fed, whereby an identical performance is achieved with less feed, or less methane is formed for the same performance. Thus, less feed and better digestibility by using the method according to the invention reduces the amount of emitted methane while maintaining the same growth capacity of the bovine.

The fermentation gas is determined as specified in the Hohenheim feeds evaluation test.

It is advantageous to employ 0.001-10 000 ppm of MOF, preferably 0.01-1000 ppm and in particular 0.1-100 ppm of MOF per kg of feed.

According to one embodiment, the porous metal-organic framework material (MOF) is magnesium formate. It is also feasible to use a magnesium formate/acetate MOF.

Advantageously, the Langmuir surface of the metal-organic framework material is at least 350 m²/g, preferably 350-500 m²/g, and in particular approximately 500 m²/g. If the specific Langmuir surface amounts to only a few square meters and is therefore too low, no effect of the metal-organic framework material can be detected.

The invention furthermore comprises a feed additive comprising at least one porous metal-organic framework material comprising at least one first and, if appropriate, one second organic compound, where at least the first organic compound binds coordinatively to at least one metal ion in an at least partly bidentate manner, where the at least one metal ion is Mg(II) and where the first organic compound is derived from formic acid and the second organic compound from acetic acid.

Surprisingly, this feed additive according to the invention makes it possible to reduce the methane produced in the digestion of feed in ruminants based on the amount of methane without the feed additive according to the invention, or to increase the total amount of gas, and therefore the energy yield, from an identical amount of feed.

According to one embodiment, the porous metal-organic framework material (MOF) is magnesium formate. It is also feasible that a magnesium formate/acetate MOF is used.

Advantageously, the Langmuir surface of the metal-organic framework material is at least 350 m²/g, preferably 350-500 m²/g and in particular approximately 500 m²/g.

Advantageously, the feed additive according to the invention is employed at a dosage rate of 0.001-10 000 ppm of MOF, preferably 0.01-1000 ppm and in particular 0.1-100 ppm of MOF per kg of feed.

### EXAMPLES

### Example 1

### Preparation of a Metal-Organic Framework Material Comprising Magnesium Formate/Acetate

Batch:

| | | | | | |
|---|---|---|---|---|---|
| 1) | Magnesium nitrate*6 H₂O | 38.5 | mmol | 9.90 | g |
| 2) | Formic acid | 53.2 | mmol | 2.5 | g |
| 3) | Acetic acid | 53.2 | mmol | 3.2 | g |
| 4) | N,N-Dimethylformamide (DMF) | 2.19 | mol | 160.0 | g |

The magnesium nitrate is dissolved in DMF in an autoclave liner. A solution of the formic acid and acetic acid is added, and the solution is stirred for 10 minutes.

Crystallization:

125° C./78 h

Product Mixture:

Clear solution with white crystals. The solution has a pH of 6.67

Work-Up:

The crystals are filtered off and washed twice with 50 ml of DMF.

Weight: 4.763 g

Solids Content:

Weight: 2.7% of solid

FIG. 1 shows the XRD of the material obtained, with I denoting the intensity (Lᵢₙ (counts)) and 2 Θ denoting the 2-theta scale.

### Example 2

### Preparation of a Metal-Organic Framework Material Based on Magnesium Formate

| | | | | | |
|---|---|---|---|---|---|
| 1) | Magnesium nitrate*6 water | 38.5 | mmol | 9.90 | g |
| 2) | Formic acid | 106.5 | mmol | 4.8 | g |
| 3) | DMF | 2.19 | mol | 160.0 | g |

The magnesium nitrate is dissolved in DMF in an autoclave liner. The formic acid is added and the solution is stirred for 10 minutes. (pH=3.49)

Crystallization:

125° C./78 h

Product Mixture:

Clear solution with white crystals

Work-Up:

The crystals are filtered off and washed twice with 50 ml of DMF.

Weight: 5.162 g

Solids Content:

Weight: 2.9% of solid

Hohenheim Feed Evaluation Test

The Hohenheim feeds evaluation test (HFT) is carried out as described in the methodological protocol of the VDLUFA (Methodenbuch volume III, chapter 25.1). By way of adaptation, the substrate weight is reduced to such an extent that a total of no more than 60 ml of gas are formed after an incubation time of 24 hours. Discharging the gas during incubation, as is usually done in the HFT, can thereby be avoided. In each case 150 mg of air-dried substance of a TMR feed are weighed in.

At the end of the experiment, the gas volume is read off, the rumen liquid/buffer mixture is immediately drained completely, and the flask sampler is resealed. Care must be taken that no air is taken in during this procedure.

The methane concentration in the fermentation gas is measured by means of an IR gas sensor for CH₄ (Advanced Gasmitter, PRONOVA Analysentechnik, 13347 Berlin). The apparatus has a measuring range of from 0 to 30% CH₄ by volume, with a display accuracy of 0.1% by volume. The analyzer is provided with an internal pressure compensation in the range of from 800 to 1200 hPa.

First, the apparatus is switched on and a preheating time of 10 minutes is allowed to elapse, whereupon the display is set to zero with the aid of the potentiometer ZERO, while passing in ambient air. Care must be taken that no negative values are displayed. The potentiometer must therefore first be turned up until the initial signal displays just 0.1% by volume. Then, it is turned down until just 0.0% by volume appear on the display. Thereafter, a test gas is passed in, the CH₄ concentration of which should correspond to the fermentation gas to be tested (15 to 20% CH₄ by volume). Now, the initial signal is adjusted to the concentration of the test gas, using the potentiometer SPAN. Finally, ambient air is passed in again, and the zero point is checked and, if appropriate, adjusted.

To prevent dirt and moisture from penetrating the sensor, a membrane filter and a small tube with a volume of 2 ml filled with a suitable absorber for steam (CaCl₂ or P₂O₅) is arranged upstream of the gas inlet. This absorber must be renewed regularly. In total, care must be taken that the dead volume between gas inlet and gas sensor is kept small in order to make do with the smallest possible amounts of sample gas. Under suitably optimized conditions, a minimum of 15 ml of sample gas are required for a reliable measurement.

To measure the fermentation gas, the outlet tube of the flask sampler is connected to the inlet of the instrument. After the tube clamp has been opened, the gas is squeezed slowly into the gas sensor. Care must be taken here that liquid is no longer present in the tube of the flask sampler; if necessary, any liquid must be removed thoroughly beforehand, using a cotton-wool bud. After at least 15 ml of gas have been passed in and the display is constant (after approximately 20 seconds), the value is read off.

The result is either presented as % CH₄ by volume or as ml CH₄ per flask sampler, by multiplying the total gas volume with the CH₄ concentration. In order to be able to better demonstrate treatment effects, an optional procedure is to determine the gas volume of the blank value (gas from rumen liquid without substrate) and its CH₄ concentration and to subtract this value from the total volume (fermentation gas or CH₄). Since the gas volume of a single blank value is not sufficient for the measurement, a bulk sample must be prepared from a plurality of blank values. As a rule, 8 replications from at least two different days are carried out for the statistic evaluation of treatment effects. The incubation time is 24 hours.

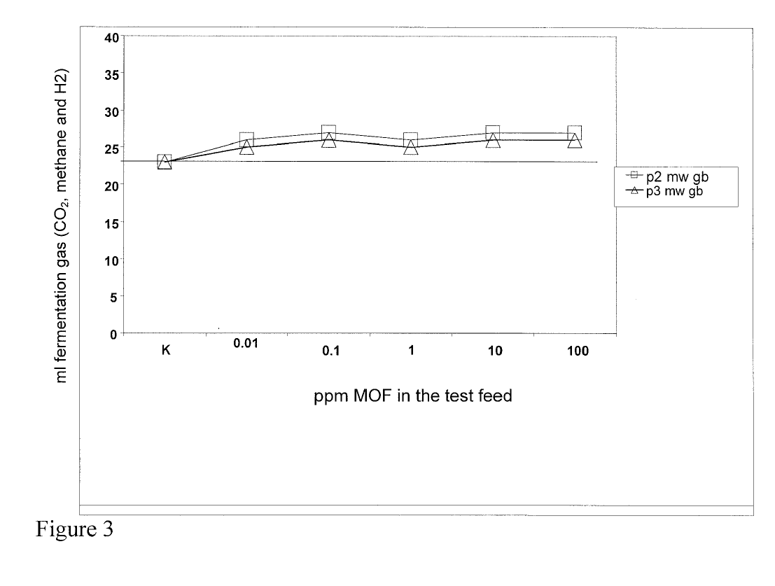

The results of the experiments are shown in FIG. 2 and FIG. 3.

FIG. 2 clearly shows a methane level in the total gas which is 10-15% lower in comparison with the control (C), independently of the amount of MOF employed. The test substance with the lower surface area value of 350 m²/g shows a markedly poorer reduction of the methane level in the gas than the substance with the higher surface area value of 500 m²/g.

FIG. 3 shows that the total gas yield from the same amount of feed is approximately 20% higher in comparison with the control (C) without additive according to the invention, or method according to the invention. Here, the total gas formation when using the substance with a slightly lower surface area value of 350 m²/g is somewhat lower than for the test substance with the surface area value of 500 m²/g. Both total gas quantities are approximately 20% above the total gas quantity of the control. The amount of the MOFs used does not result in any significant difference in the amount of the gas formed in total. The total gas formed comprises predominantly CO₂, methane and small amounts of hydrogen.

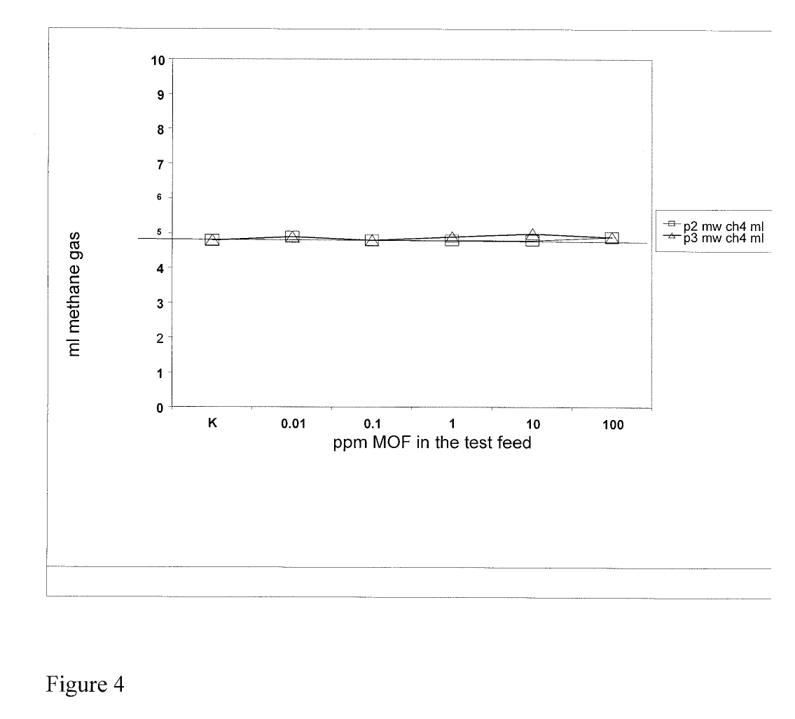

FIG. 4 shows that, with a constant amount of feed, the amount of methane produced remains constant while the feed conversion rate is better by using the MOF magnesium formates, or the method according to the invention.

No increased amount of methane is liberated, while the energy yield for the animal is better and, as a consequence, its growth is more rapid. As an alternative, the use according to the invention, or the method according to the invention, and the feed additive according to the invention allows the amount of methane produced to be achieved with less feed, with the same performance, since the total energy yield from the feed is increased by the use according to the invention.

We claim:

1. A method of reducing the methane level in the total gas produced in ruminants during the feed digestion, comprising the feeding, to a ruminant, of at least one porous metal-organic framework material (MOF) comprising at least one first and optionally one second organic compound, where at least the first organic compound binds coordinatively to at least one metal ion in an at least partly bidentate manner, where the at least one metal ion is Mg(II) and where the first organic compound is derived from formic acid and the second organic compound from acetic acid.

2. The method according to claim 1, wherein the amount of MOF per kg of feed is 0.001-10 000 ppm.

3. The method according to claim 1, wherein the amount of MOF per kg of feed is 0.01-1000 ppm.

4. The method according to claim 1, wherein the amount of MOF per kg of feed is 0.1-100 ppm.

5. The method according to claim 1, wherein the MOF is magnesium formate.

6. The method according to claim 1 wherein the Langmuir surface of the MOF is at least 350 m²/g.

7. The method according to claim 1, wherein the Langmuir surface of the MOF is 350 to 500 m²/g.

8. A method of increasing the total gas formation in ruminants during the feed digestion, comprising the feeding, to a ruminant, of at least one porous metal-organic framework material (MOF) comprising at least one first and, optionally one second organic compound, where at least the first organic compound binds coordinatively to at least one metal ion in an at least partly bidentate manner, where the at least one metal ion is Mg(II) and where the first organic compound is derived from formic acid and the second organic compound from acetic acid.

9. The method according to claim 8, wherein the amount of MOF per kg of feed is 0.001-10 000 ppm.

10. The method according to claim 8, wherein the amount of MOF per kg of feed is 0.01-1000 ppm.

11. The method according to claim 8, wherein the amount of MOF per kg of feed is 0.1-100 ppm.

12. The method according to claim 8, wherein the MOF is magnesium formate.

13. The method according to claim 8, wherein the Langmuir surface of the MOF is at least 350 m²/g.

14. The method according to claim 8, wherein the Langmuir surface of the MOF is 350 to 500 m²/g.

15. A feed additive comprising at least one porous metal-organic framework material (MOF) comprising at least one first and optionally one second organic compound, where at least the first organic compound binds coordinatively to at least one metal ion in an at least partly bidentate manner, where the at least one metal ion is Mg(II) and where the first organic compound is derived from formic acid and the second organic compound from acetic acid.

16. The feed additive according to claim 15, wherein the MOF is magnesium formate.

17. The feed additive according to claim 15, wherein the Langmuir surface of the MOF is at least 350 m²/g,

18. The feed additive according to claim 15, wherein the Langmuir surface of the MOF is 350 to 500 m²/g.

19. The feed additive according to claim 15, wherein the amount of MOF per kg of feed is 0.01-1000 ppm.
